(19) United States
(12) Patent Application Publication
(10) Pub. No.: US 2003/0021749 A1
(43) Pub. Date: Jan. 30, 2003

(54) Treatment of cancer and other diseases by administration of positron-emitting radiopharmaceuticals

(76) Inventor: Alex Wah Hin Yeung, Hong Kong (CN)

Correspondence Address: MORRISON & FOERSTER, LLP, 555 WEST FIFTH STREET, SUITE 3500, LOS ANGELES, CA, 90013-1024, US

(21) Appl. No.: 10/020,368

(22) Filed: Dec. 11, 2001

Related U.S. Application Data
(60) Provisional application No. 60/256,671, filed on Dec. 18, 2000.

(51) Int. Cl. A61K 51/00

(52) U.S. Cl. 424/1.73; 424/1.65

(57) ABSTRACT

Fluorine-18, fluorine-18-incorporating radiopharmaceuticals (FRPs), and other positron-emitting isotopes and radiopharmaceuticals have been used for diagnostic purposes in positron emission tomography (PET). This invention describes the therapeutic use of positron-emitting radiopharmaceuticals such as FRPs primarily, but not exclusively, for the treatment of cancer and cancer-related illnesses. The radiopharmaceuticals are administered in dosages significantly higher than those used for diagnostic imaging.

### CROSS-REFERENCE TO RELATED APPLICATIONS

This application claims priority benefit of United States Provisional Patent Application Serial No. 60/256,671, filed Dec. 18, 2000. The contents of that application are hereby incorporated by reference herein in their entirety.

### STATEMENT OF RIGHTS TO INVENTIONS MADE UNDER FEDERALLY SPONSORED RESEARCH

Not applicable.

### TECHNICAL FIELD

This invention relates to novel therapeutic uses of fluorine-18, fluorine-18 labeled compounds, and other isotopically-labeled pharmaceuticals and radiopharmaceuticals for treatment of human diseases, particularly for treatment of cancer.

### BACKGROUND ART

Positron emission tomography (PET) is a widely used technique for imaging. PET scanning operates by administration of a positron-emitting compound to the subject to be scanned. This compound then localizes in various tissues in the body. When the compound decays by positron emission, the emitted positron will encounter an electron. The resulting matter-antimatter annihilation event results in the production of a pair of photons of approximately 511 keV which are detected by the PET scanner (10).

Typically the positron-emitting compound has a relatively short half-life, and must be produced as soon as possible prior to administration to the subject. Radioisotopes are generated at the treatment facility by cyclotron radiation, and can then be chemically incorporated into the compound for administration. The fluorine-18 (¹⁸F) isotope is a widely used radioisotope for incorporation into compounds for PET scanning, due to its relatively long half life of 110 minutes. This allows sufficient time for synthesis of the labeled compound, administration to the subject, and uptake by the tissues of interest. Typical dosages of fluorine-18 containing compounds for PET scanning range from 3 to 20 mCi per 50 kg of body weight.

A widely used compound for PET scanning is fluorodeoxyglucose (FDG). FDG has been used for over 10 years for detection and monitoring of cancer, and provides advantages over other existing imaging modalities for imaging of colorectal cancer, lung cancer, melanoma, lymphoma, and breast cancer. FDG has been used in PET scanning for metabolic imaging of cancer, heart disease, and neurological disease.

It has now been discovered that diseases can be treated by administration of radiolabeled positron-emitting compounds in dosages significantly higher than those used for diagnostic and imaging purposes. The current invention provides methods for treatment of disease using positron-emitting compounds.

### DISCLOSURE OF THE INVENTION

The invention provides new methods for treating disease in subjects, particularly cancer and related diseases.

In one embodiment, the invention provides a method of treating a disease in a subject, comprising administering a therapeutically effective amount of a positron-emitting compound to the subject. The positron emitting compound can comprise one or more atoms of fluorine-18, carbon-11, nitrogen-13, or oxygen-15. In another embodiment, the positron-emitting compound comprises one or more atoms of fluorine-18.

In one embodiment, the positron-emitting compound is ¹⁸F-fluorodeoxyglucose. In another embodiment, the ¹⁸F-fluorodeoxyglucose is ¹⁸F-2-fluoro-2-deoxyglucose. In yet another embodiment, the positron-emitting compound is ¹⁸F-fluorocholine. In a further embodiment, the positron-emitting compound is [methyl-¹¹C] choline.

In one embodiment of the invention, the positron-emitting compound is administered to the subject in a dosage at least about 1.5 to 2 times that used for diagnostic purposes. In another embodiment, the positron-emitting compound is administered to the subject in the maximum dosage that the patient can tolerate. In another embodiment, the positron-emitting compound is administered in a dosage of about 30 to 100 mCi per 50 kg of body weight. In another embodiment, the dosage of the positron emitting compound is about 30 mCi per 50 kg of body weight. In another embodiment, the dosage is about 40 mCi per 50 kg of body weight. In another embodiment, the dosage is about 50 mCi per 50 kg of body weight. In another embodiment, the dosage is about 60 mCi per 50 kg of body weight. In another embodiment, the dosage is about 70 mCi per 50 kg of body weight. In another embodiment, the dosage is about 80 mCi per 50 kg of body weight. In another embodiment, the dosage is about 90 mCi per 50 kg of body weight. In another embodiment, the dosage is about 100 mCi per 50 kg of body weight. In another embodiment, the positron-emitting compound administered in the preceding dosages is ¹⁸F-fluorodeoxyglucose. In another embodiment, the positron-emitting compound administered in the preceding dosages is ¹⁸F-2-fluoro-2-deoxyglucose.

In another embodiment of the invention, the positron-emitting compound can be administered in conjunction with any combination of immunotherapy, surgery, radiation therapy, or other chemotherapy to the subject at any stage in the treatment of the subject.

In a further embodiment of the invention, the positron-emitting compound is administered intravenously.

In any of the foregoing methods, the positron-emitting compound can be administered in one or two dosages per day, for between two to ten days sequentially or non-sequentially over a two-week to three-week period. In another embodiment, in any of the foregoing methods, the positron-emitting compound can be administered in one or two dosages per day, for between five to ten days sequentially or non-sequentially over a two-week to three-week period. In one embodiment of the invention, between five and ten dosages are administered on consecutive days. In another embodiment of the invention, between five and ten dosages are administered on non-consecutive days over a two-week to three-week period. In another embodiment of the invention, between five and ten dosages are administered on non-consecutive days over a two-week period.

In one embodiment of the invention, the disease to be treated is cancer. In yet another embodiment, the disease is bone cancer.

In further embodiments, the invention embraces a composition comprising a dosage of any of the compounds described herein that is at least 1.5 to 2 times as high as the dosage used for diagnostics applications. The invention also embraces use of a medicament comprising any of the compounds described herein for the preparation of a medicament for therapeutic treatment of cancer. The invention also embraces unit dosage preparations of the compounds for therapeutic treatment of cancer, in any of the dosages described herein as useful for therapeutic treatment of cancer.

### DETAILED DESCRIPTION OF THE INVENTION

### Definitions

“Treating” a disease or pathology is defined as preventing, eliminating, reducing the severity of, mitigating, or preventing the further development or spread of a disease or pathology, or preventing, eliminating, reducing the severity of, mitigating, or preventing the further development of the symptoms of a disease or pathological state. “Treating” a subject, individual, or patient is defined as treating the disease afflicting the subject, individual, or patient.

A “therapeutically effective amount” of a compound is a dosage of that compound sufficient for preventing, eliminating, reducing the severity of, mitigating, or preventing the further development or spread of a disease or pathology, or preventing, eliminating, reducing the severity of, mitigating, or preventing the further development of the symptoms of a disease or pathological state.

By “individual,” or “subject” is meant a vertebrate, preferably a mammal, more preferably a human. “Patient” refers to an individual or subject who has undergone, is undergoing, or will undergo treatment.

By “administration” is meant introducing a compound into a subject. The preferred route of administration of the compounds is intravenous. However, any route of administration, such as oral, topical, subcutaneous, peritoneal, intraarterial, inhalation, vaginal, rectal, nasal, introduction into the cerebrospinal fluid, or instillation into body compartments can be used.

### Methods of the Invention

The present invention relates to novel therapeutic uses of positron-emitting radiopharmaceuticals, such as fluorine-18 containing compounds, in human diseases. The methods of the invention are particularly useful in the treatment of cancer and related diseases.

Compounds useful in the invention are those compounds incorporating radioisotopes which emit positrons. Radioisotopes which emit positrons include, but are not limited to, fluorine-18 (half-life of 110 minutes), carbon-11 (half-life of 20 minutes), nitrogen-13 (half-life of 10 minutes), and oxygen-15 (half-life of 2 minutes). Compounds into which positron-emitting radioisotopes can be incorporated include, but are not limited to, proteins, such as monoclonal and polyclonal antibodies and enzymes; linear, cyclic, and branched peptides, including N-methylated peptides and peptides incorporating D-amino acids or non-naturally occurring amino acids; peptidomimetics, such as peptoids, peptide nucleic acids, and peptides where the peptide bond is reduced or otherwise stabilized against hydrolysis; amino acids, including the twenty amino acids encoded by the genetic code, and other naturally occurring amino acids and non-naturally occurring amino acids (including, but not limited to, peptide nucleic acids, D-amino acids, amino acid alcohols, acetylated amino acids, other acylated amino acids, amino acid esters, amino acid amides, beta-amino acids, peptide-nucleic acids, and the like); nucleic acids, including, but not limited to, DNA, RNA, nucleotides, nucleosides, nucleotide analogs and nucleoside analogs; carbohydrates, including, but not limited to, glucose, deoxyglucose, fluorodeoxyglucose, 2-fluoro-2-deoxyglucose, fructose, sucrose, galactose, lactose, and the like; drugs, such as chemotherapeutic agents; small organic molecules, preferably with a molecular weight of less than 1000, more preferably with a molecular weight of less than 600, including, but not limited to, choline, fluorocholine, and acetylcholine; and substrates for biochemical reactions occurring in the subject.

A compound useful in the invention is ¹⁸F-fluorodeoxyglucose (referred to as fluoro-18 FDG, ¹⁸F-FDG, or simply FDG). Generally, ¹⁸F-2-fluoro-2-deoxyglucose is used. (The synthesis of this compound has been published and is known to those of skill in the art.) The FDG is administered to the subject, preferably via intravenous injection. Following intravenous administration of FDG, most of the radiopharmaceutical is rapidly cleared from the circulation with a half-life of less than 1 minute, as it mixes within a large distribution space, although there are longer-term compartments with half lives of up to 1.5 hours. The substrate is taken up predominantly by malignant tumor tissues, infectious tissues, the myocardium, and the brain. There is evidence from investigations on dogs (7) of concentration in other organs, especially the spleen, liver, and kidneys, but significant uptake in these organs has not been observed in human studies (9). Other compounds useful for the methods of the invention include fluorine-18 fluorocholine and [¹¹C methyl]choline.

The therapeutic dosage of FDG for use in the methods of the invention is at least 30 mCi per 50 kg of body weight per dose. The therapeutic dosage is generally between 30 and 100 mCi per 50 kg of body weight. Thus, other useful dosages are 30, 40, 50, 60, 70, 80, 90, and 100 mCi per 50 kg of body weight per dose.

Approximately 20% of the administered fluorine-18 is excreted in urine within the first 2 hours (8). From the average urine data of Jones et al. (8), it can be deduced that the total body retention of FDG may be described, for purposes of dosimetry, by a multiexponential function with a halftime of 12 min. (0.075), 1.5 hour (0.225), and infinity (0.70). Fractions of 0.04 and 0.06 are taken up by myocardium and brain, respectively, with an uptake half time of 8 minutes, and retained for a time which is long in relation to the radioactive half life of fluorine-18. The residual activity in the total body is assumed to be uniformly distributed amongst all tissues other than the brain and heart. A fraction of 0.3 is assumed to be eliminated by the renal system with half times of 12 minutes (0.25) and 1.5 hours (0.75) according to the kidney-bladder model.

Extrapolating from this bio-kinetic model, the PET scan estimated dose equivalent (EDE) is about 1 rem for normal tissues with fluoro-18 FDG at a dosage of 10 mCi. For a malignant tumor, assuming a 1 cm tumor diameter and the same 10 mCi dose, the EDE would be 137.5 rems with a 1% uptake and up to 550.1 rems with a 4% tumor uptake (6). Generally, the size of the tumor and the uptake percent govern the exact dose absorbed by the malignant tissue.

From this calculation, with an FDG dosage of 30 mCi, which is three times a typical 10 mCi dosage used for diagnostic purposes, a 1 cm tumor with a 4% uptake will receive an EDE of 1,650 rems. Such a 30 mCi dosage of FDG can be administered once or twice a day for five to ten days (either on consecutive days, or on non-consecutive days, that is, at intervals over a course of time), with correspondly increased cumulative EDE's. The cumulative EDE can be adjusted by altering the dosage appropriately. These calculations assume that there is no loss of FDG absorption due to tumor regression or other factors.

Toxicity to major organs, such as the brain and the heart, will remain relatively low because of the large size of the heart and brain tissue relative to the tumor tissue. For the myocardium, the normal EDE of 2.2 rems will be increased to 33 rems after a therapeutic course with 5 fractions of 30 mCi of FDG. Brain and myocardial damage from one course of such treatment is thus estimated to be minimal. Care should be taken to adjust the dosage appropriately if the patient has previously received radiation treatment of those organs, in order to prevent significant damage. According to a widely used international safety standard, the dose limit for any single organ is 50 rems yearly for a radiation worker (4). However, this figure can be relaxed if the procedure is for treatment purposes.

Toxicity to the brain can be further reduced by using other fluoro-18 labeled compounds, such as fluorine-18 fluorocholine (FCH). FCH does not accumulate in normal brain tissue as readily as FDG, and has the additional advantage of achieving higher concentration in tumor tissues (1, 2, 3, 5). [Methyl-¹¹C] choline can also be used in the methods of the invention.

The invention thus provides straightforward methods for delivering a therapeutic agent to malignant tissue, with resulting damage to the malignant tissue and minimal damage to healthy tissue. The methods of the invention can be used either alone or in combination with traditional methods of cancer therapy such as radiation therapy, chemotherapy, immunotherapy, and surgery. The invention thus provides useful procedures for the treatment of cancer and other diseases.

### EXAMPLE 1

### Case History

A sixty-six year old man complained of left hip pain for six months. He had a history of renal cell carcinoma with renal vein invasion two years ago. Nephrectomy was performed and he was given a course of alpha-interferon for six months. During the current episode, MRI showed a large solitary left iliac metastasis about 7 cm in diameter.

### Treatment with Fluoro-18 FDG

Two treatments with 30 mCi of fluoro-18 FDG were given three weeks apart. No untoward side effects were noted after the administration by injection. At the end of six weeks, significant reduction of both the size of the lesion and subjective symptoms were achieved. At this juncture, the patient was treated with definitive external beam irradiation. Six months later, the patient remained symptom free and his pelvic lesion had healed.

### PET Scanning Results

Before and during FDG treatments, PET scans were performed. Progressive reduction of the standard uptake value was achieved after each treatment and after the definitive external beam irradiation.

### Conclusion

By the predominantly gamma radiation from the positron emission of the fluoro-18 FDG which accumulated in the solitary pelvic metastasis from the renal carcinoma, a positive response was achieved after two treatments. Bone cancer secondary from renal cell carcinoma is notoriously difficult to treat; the disease is essentially resistant to any chemotherapy or immunotherapy currently available. By achieving a positive response with the fluoro-18 FDG treatment followed by definitive external beam irradiation, this patient is expected to achieve a relatively long progression free and symptom free survival period with good quality of life. This outcome would have been very difficult to achieve with other treatments given the history of his case.

### REFERENCES

1. Roivainen A. et al., “Blood metabolism of [methyl-¹¹C]choline; implications for in vivo imaging with positron emission tomography,” Eur. J. Nucl. Med. 27(1):25-32 (January 2000).

2. Kobori O. et al., “Positron emission tomography of esophageal carcinoma using ¹¹C-choline and ¹⁸F-fluorodeoxyglucose: a novel method of preoperative lymph node staging,” Cancer 86(9):1638-48 (Nov. 1, 1999).

3. Hara T. et al., “PET imaging of brain tumor with [methyl-¹¹C]choline,” The Journal of Nuclear Medicine, 38(6):842-7 (June 1997).

4. Laws of the Hong Kong Government: Radiation (Control of Irradiating Apparatus), Regulations on, (Cap. 303, Section 13) (Oct. 1, 1965), L. N. 114 of 1965.

5. Coleman R. E. et al., “Preliminary Evaluation of F-18 Fluorcholine (FCH) as a PET Tumor Imaging Agent,” Scientific Paper from 12th Annual International PET Conference, Oct. 15-18, 2000, Washington, D.C.

6. Calculation by Vanessa Gates, M S, and C. Oliver Wong, MBBS, PhD, MD, FACP, FACNP, Ohio State University and Michigan State University.

7. Gallagher, B. M. et al., “Radiopharmaceuticals XXVII: 18F-labelled 2-deoxy-2-fluoro-D-glucose as a radiopharmaceutical for measuring myocardial glucose metabolism in vivo. Tissue distribution and imaging studies in animals,” J. Nucl. Med. 18:990-996 (1977).

8. Jones S. C. et al., “The radiation dosimetry of 2-F-18 fluoro-2-deoxy-D-glucose in man,” J. Nucl. Med. 23:613-617 (1982).

9. Phelps, M. E. et al., “Investigation of [18F] 2-fluoro-2-deoxyglucose for the measure of myocardial glucose metabolism,” J. Nucl. Med. 19:1311-1319 (1978).

10. Gopal B. Saha et al., “Cyclotrons and Positron Emission Tomography Radiopharmaceuticals for Clinical Imaging,” Seminars in Nuclear Medicine, Vol. XXII, No. 3 (July 1992), pp. 150-161.

All patents, patent applications, and other publications mentioned herein are hereby incorporated by reference herein in their entirety.

Although the foregoing invention has been described in some detail by way of illustration and example for purposes of clarity and understanding, it will be apparent to those skilled in the art that certain changes and modifications may be practical. Therefore, the description and examples should not be construed as limiting the scope of the invention, which is delineated by the appended claims.

What is claimed is:

1. A method of treating a disease in a subject, comprising:
administering a therapeutically effective amount of a positron-emitting compound to the subject, wherein the positron emitting compound comprises one or more atoms of fluorine-18, carbon-11, nitrogen-13, or oxygen-15.

2. The method of claim 1, wherein the positron-emitting compound comprises one or more fluorine-18 atoms.

3. The method of claim 2, wherein the positron-emitting compound is ¹⁸F-fluorodeoxyglucose.

4. The method of claim 3, wherein the ¹⁸F-fluorodeoxyglucose is ¹⁸F-2-fluoro-2-deoxyglucose.

5. The method of claim 2, wherein the positron-emitting compound is ¹⁸F-fluorocholine.

6. The method of claim 1, wherein the positron-emitting compound is [methyl-¹¹C] choline.

7. The method of claim 1, wherein the positron-emitting compound is administered to the subject in a dosage at least about 1.5 times that used for diagnostic purposes.

8. The method of claim 7, wherein the positron-emitting compound is administered to the subject in a dosage at least about twice that used for diagnostic purposes.

9. The method of claim 1, wherein the positron-emitting compound is administered to the subject in a dosage of about 30 to 100 mCi per 50 kg of body weight.

10. The method of claim 1, wherein the positron-emitting compound is administered to the subject in a dosage of about 30 mCi per 50 kg of body weight.

11. The method of claim 1, wherein the positron-emitting compound is administered to the subject in a dosage of about 50 mCi per 50 kg of body weight.

12. The method of claim 1, wherein the positron-emitting compound is administered to the subject in a dosage of about 100 mCi per 50 kg of body weight.

13. The method of claim 1, further comprising administering any one of or any combination of immunotherapy, surgery, or other chemotherapy or radiation therapy to the subject at any stage in the treatment of the subject.

14. The method of claim 1, wherein the positron-emitting compound is administered intravenously.

15. The method of claim 1, wherein the positron-emitting compound is administered in one or two doses per day over five to ten days during a two-week to three-week period.

16. The method of claim 4, wherein the positron-emitting compound is administered in one or two doses per day over five to ten days during a two-week to three-week period.

17. The method of claim 15, wherein the doses are administered on five to ten consecutive days.

18. The method of claim 15, wherein the doses are administered on non-consecutive days.

19. The method of claim 1, wherein the disease is cancer.

20. The method of claim 19, wherein the cancer is bone cancer.
